(19) United States
(12) Patent Application Publication
(10) Pub. No.: US 2005/0106187 A1
(43) Pub. Date: May 19, 2005

(54) Administration form for the oral administration of active substances, vitamins and/or nutrients

(76) Inventor: Rasoul Sedaghat Kerdar, Aachen (DE)

Correspondence Address: Geza C. Ziegler, Jr.;PERMAN & GREEN, LLP., 425 Post Road, Fairfield, CT, 06824, US

(21) Appl. No.: 11/021,643

(22) Filed: Dec. 23, 2004

Related U.S. Application Data
(63) Continuation of application No. PCT/EP03/06533, filed on Jun. 20, 2003.

(30) Foreign Application Priority Data
Jun. 24, 2002 (DE) 102 28 176.9

(51) Int. Cl. A61K 9/00; A61K 9/20; E03B 9/20; B05B 12/14

(52) U.S. Cl. 424/400; 604/500; 239/24

(57) ABSTRACT

The invention relates to an administration form for orally administering active substances, vitamins, and/or nutrients, including a drinking straw that is provided with a cap. The cap includes a pierceable bottom that is made of an optionally laminated aluminum-plastic film or cellulose film or a shock-proof plastic bottom which is removable by a pushing movement and is at least partly recessed via a weakening line as a sealing device. Also disclosed are a kit including the administration form and a conveying liquid, and a method for producing the administration form.

The invention relates to a dosage form for the oral administration of active ingredients, vitamins and/or nutrients comprising a drinking straw with a cap with a pierceable bottom made of an optionally laminated aluminium foil, plastics or cellulose film or with an impact-resistant plastics bottom which is removable by impact and is at least partially recessed via a weakening line as a closing device, to a kit comprising the dosage form and a conveying liquid and to a process for the production of the dosage form.

It is known to administer active ingredients, vitamins and/or nutrients with the assistance of a drinking straw which contains an active ingredient, vitamin and/or nutrient formulation which is conveyable by a liquid. Patients who have difficulty in or an aversion to taking capsules or tablets are provided in this manner with a further dosage form for active ingredients, vitamins or nutrients.

The drinking straws described in the prior art for the oral administration of active ingredients, vitamins and/or nutrients comprise bungs or caps as a closing device in order to retain the active ingredient, vitamin and/or nutrient formulation located in the drinking straw. The bungs or caps must be taken off the drinking straw before the drinking straw is used. There is a risk of spilling the active ingredient, vitamin and/or nutrient formulation while these known closing devices are being removed.

The object therefore arose of providing a dosage form for the oral administration of active ingredients, vitamins and/or nutrients comprising a drinking straw with a closing device, which dosage form is as simple as possible to produce and does not exhibit the disadvantages of the dosage forms described in the prior art.

This object is achieved according to the invention by the provision of a dosage form for the oral administration of active ingredients, vitamins and/or nutrients comprising a drinking straw, which has two orifices and contains a barrier device which is impermeable to an active ingredient, vitamin and/or nutrient formulation, but is permeable to air and a conveying liquid, a closing device which is impermeable to the active ingredient, vitamin and/or nutrient formulation, and the active ingredient, vitamin and/or nutrient formulation arranged in the drinking straw between the barrier device and the closing device, characterised in that, as the closing device, a cap which closes the drinking straw orifice facing the formulation comprises a pierceable bottom made from aluminium foil or an aluminium/plastics film laminate, a single layer or multilayer plastics film or cellulose film or is equipped with an impact-resistant bottom made of plastics or metal, preferably aluminium, which is removable by impact and is at least partially connected via a weakening line.

At least one end of the drinking straw may be provided with a removable protective cap, whose bottom which is not removable by impact consists of an impact-resistant material.

The cap used according to the invention may be produced by producing a tube, preferably of plastics, with an appropriate diameter for enclosing at least the mouthpiece of the drinking straw and connecting the bottom made from the desired foil/film therewith. This foil/film may optionally also comprise weakening lines to facilitate piercing.

The closing cap may also, for example, overall be manufactured from the same material, preferably plastics or metal, such as aluminium, optionally laminated for reinforcement, the bottom of which comprises a weakening line at least partially around the entire outer periphery. In this manner, the otherwise impact-resistant bottom may be removed by impact.

The active ingredient, vitamin and/or nutrient formulation is preferably solid or pasty. A multiparticulate active ingredient, vitamin and/or nutrient formulation is preferred, wherein the particles of the multiparticulate formulation preferably have a diameter of 50-1500 μm.

The active ingredients, vitamins and/or nutrients alone or the overall active ingredient, vitamin and/or nutrient formulations may be insoluble or at least partially soluble in the conveying liquid.

If the active ingredients, vitamins or nutrients or the formulations thereof are soluble in the conveying liquid, they are dissolved by the conveying liquid as it flows past and entrained in dissolved form. If, on the other hand, the active ingredients, vitamins or nutrients or the formulations thereof are insoluble in the conveying liquid, they are suspended in the conveying liquid and entrained with the conveying liquid in suspended form. Multiparticulate formulations are preferably used for this purpose. The particles should be so small and light that they can be conveyed by the conveying liquid. Adhesion of the particles to one another or to the walls of the drinking straw should be as slight as possible, which may optionally be achieved by a suitable surface treatment of the particles.

Active ingredients, preferably therapeutic active ingredients, vitamins and nutrients which may be used are any orally administrable substances in a suitable formulation. The drinking straw preferably contains a precisely determined dose of these substances.

The barrier device preferably takes the form of a plug, the cross-section of which is preferably adapted to the cross-section of the drinking straw. The uninserted plug may, however also have a larger cross-section, provided that this plug can be compressed to a cross-section corresponding to the internal cross-section of the drinking straw.

The barrier device may be permanently fixed in the drinking straw or be arranged movably. The plug may accordingly, on the one hand, be permanently fixed in the drinking straw, by fixing the plug, for example, by narrowing the drinking straw or by adhesively bonding the plug in the drinking straw. The plug is preferably fixed by narrowing the drinking straw.

The plug may, on the other hand, also be movable by the conveying liquid between two limit stops. The drinking straw is narrowed by these limit stops, wherein the narrowed portions are designed such that the plug is retained thereby, but the formulation particles insoluble in the conveying liquid are not.

The plug preferably consists of filter material, particularly preferably of a nonwoven material or a compressed fibrous material.

A membrane fixed in the drinking straw is also preferred as a barrier device, wherein the cross-section of the membrane is preferably adapted to the cross-section of the drinking straw and the membrane is preferably adhesively bonded in the drinking straw. The membrane preferably consists of a synthetic material.

Another preferred barrier device is a mesh which is fixed in the drinking straw and has a mesh diameter smaller than the diameter of the smallest formulation particle. The cross-section of the mesh is preferably adapted to the cross-section of the drinking straw and the mesh is preferably fixed in the drinking straw by narrowing of the drinking straw or is adhesively bonded in the drinking straw. The mesh preferably consists of synthetic material.

A narrowing of the drinking straw which is designed such that even the smallest formulation particles are retained thereby is also suitable as a barrier device.

The barrier device must not reduce the flow of the conveying liquid to such an extent that a solid, insoluble active ingredient, vitamin and/or nutrient formulation can no longer be entrained.

The drinking straw may be rigid or flexible, straight, preferably reversibly bendable, or angled.

The drinking straw preferably consists of a synthetic material. At least the optionally present zone in which the drinking straw is reversibly bendable is preferably produced from a resilient synthetic material or a synthetic material which is plastically deformable at a temperature below the softening point thereof. The structure of the zone in which the drinking straw is reversibly bendable preferably resembles an accordion.

The internal diameter of the drinking straw should preferably be at least 3 mm such that a sufficient quantity of conveying liquid to convey the formulation can be sucked through the drinking straw. An internal diameter of 4-15 mm is preferred, an internal diameter of 5-10 mm being very particularly preferred.

The drinking straw preferably has a round, oval, rectangular or square cross-section and the inside thereof is preferably smooth.

The drinking straw orifice facing the formulation preferably assumes the form of a mouthpiece, particularly preferably ribbed, while the drinking straw orifice facing the barrier device preferably takes the form of a connecting piece to a reservoir of the conveying liquid. The drinking straw is preferably labelled to indicate the mouth part and/or to indicate the direction of sucking for the conveying liquid. Such marking simplifies handling.

At least the part of the drinking straw containing the formulation is transparent and/or coloured. When a transparent drinking straw is used, uptake of the active ingredient, vitamin and/or nutrient formulation may be observed, while using an opaquely coloured drinking straw makes it possible to conceal the administration of an active ingredient, vitamin and/or nutrient formulation.

The present invention also provides a kit comprising the dosage form according to the invention and a physiologically unproblematic conveying liquid which optionally contains active ingredients, nutrients and/or vitamins.

A suitable conveying liquid is preferably an aqueous liquid, wherein water, lemonade, fruit juice without fruit pulp, tea or coffee are particularly preferred.

The present invention also provides a process for the production of the dosage form according to the invention, in which the barrier device is inserted into the drinking straw, the formulation is introduced into the drinking straw through one drinking straw orifice and the drinking straw orifice facing the formulation is provided with the closing device.

The following Figures show exemplary embodiments of the dosage forms according to the invention, but this does not constitute any limitation of the concept of the invention.

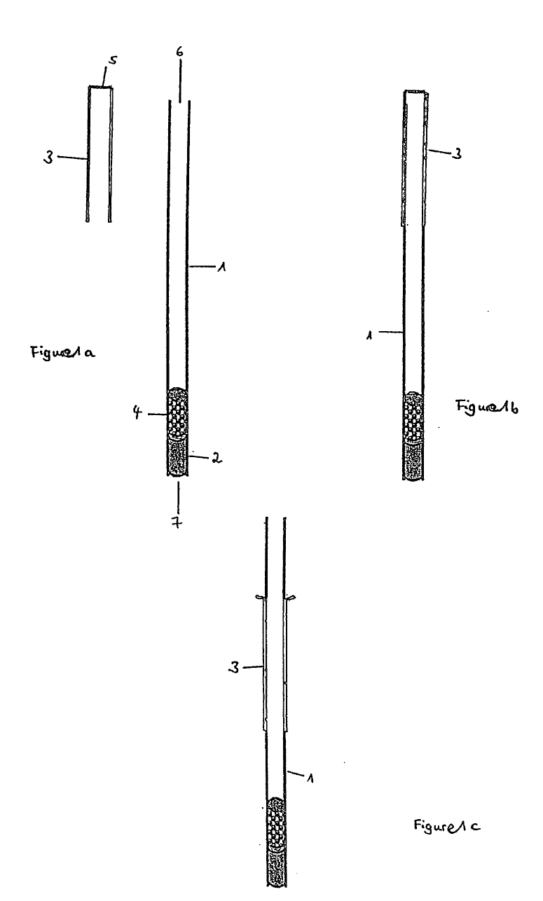

FIGS. 1a, 1b and 1c show a side view of a dosage form according to the invention with a removed cap with a pierceable bottom (a), a cap in place with a pierceable bottom (b) and a cap with a pierced bottom (c).

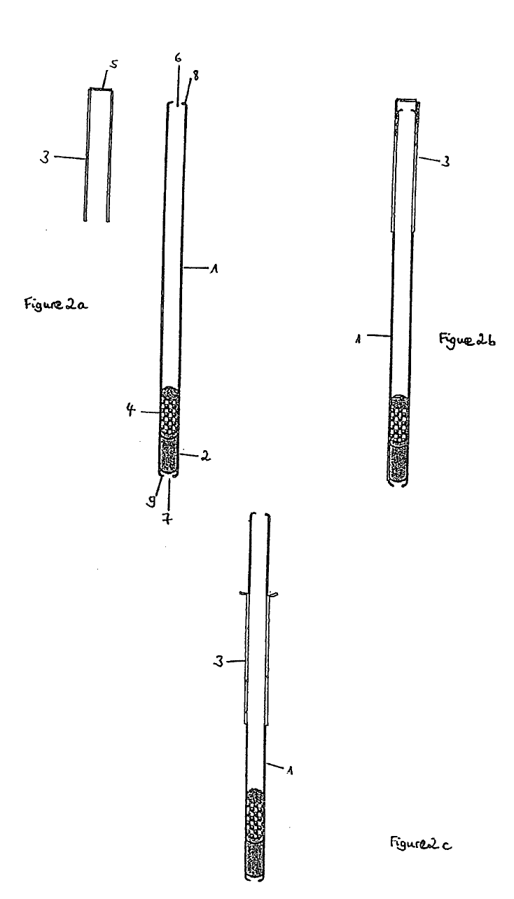

FIGS. 2a, 2b and 2c show a side view of a further dosage form according to the invention with a removed cap with a pierceable bottom (a), a cap in place with a pierceable bottom (b) and a cap with a pierced bottom (c).

The dosage form shown in FIGS. 1a, 1b and 1c comprises a drinking straw (1), which has two orifices (6, 7) and a plug fixed in the drinking straw as a barrier device (2), a cap with a pierceable bottom (5) as a closing device (3) and a formulation (4) located in the drinking straw between the barrier device and the closing device.

The drinking straw (1) has a round cross-section and is straight.

The plug (2) serving as a barrier device consists of filter material and is permeable to air and the conveying liquid, but impermeable to the formulation. The plug is cylindrical and has a round cross-section, wherein the diameter of the plug is adapted to the internal diameter of the drinking straw. The plug is permanently fixed in the drinking straw.

The formulation (4) assumes the form of spherical particles.

The cap (3) serving as a closing device is impermeable to the formulation. The internal diameter of the cap (3) is adapted to the external diameter of the drinking straw (1) such that the cap may be placed on the drinking straw. Before the drinking straw (1) is used, the bottom (5) of the cap (3) is pierced with the assistance of the drinking straw (1). The cap (3) with broken bottom (5) is drawn over the drinking straw.

The dosage form shown in FIGS. 2a, 2b and 2c substantially corresponds to the dosage form shown in FIGS. 1a, 1b and 1c. Instead of a fixed plug, however, a plug which is movable by the conveying liquid between two limit stops (8, 9) is used as the barrier device (2).

The diameter of the largest formulation particles is less than the internal diameter of the drinking straw at the limit stop (8), such that the formulations particles can pass through this point.

1. A dosage form for the oral administration of active ingredients, vitamins and/or nutrients comprising a drinking straw (1), which has two orifices (6, 7) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation, and the active ingredient, vitamin and/or nutrient formulation (4) arranged in the drinking straw between the barrier device and the closing device, characterised in that, as the closing device, a cap (3) which closes the drinking straw orifice (6) facing the formulation comprises a bottom (5) plastics or metal, preferably aluminium, which is removable by impact and comprises a weakening line at least partially around the entire outer periphery thereof.

2. (canceled)

3. A dosage form according to claim 1, characterised in that the active ingredient, vitamin and/or nutrient formulation (4) is solid.

4. A dosage form according to claim 1, characterised in that the active ingredient, vitamin and/or nutrient formulation (4) is pasty.

5. A dosage form according to claim 1, characterised in that the active ingredient, vitamin and/or nutrient formulation (4) is in multiparticulate form.

6. A dosage form according to claim 1, characterised in that the active ingredient, vitamin and/or nutrient formulation (4) is insoluble in the conveying liquid.

7. A dosage form according to claim 1, characterised in that the active ingredient, vitamin and/or nutrient formulation (4) is at least partially soluble in the conveing liquid.

8. A dosage form according to claim 1, characterised in that the barrier device (2) is a plug.

9. A dosage form according to claim 8, characterised in that the plug (2) is movable by the conveying liquid between two limit stops (8, 9).

10. A dosage form according to claim 8, characterised in that the plug (2) is fixed.

11. A dosage form according to claim 1, characterised in that the barrier device (2) is a membrane fixed in the drinking straw.

12. A dosage form according to claim 5, characterised in that the barrier device (2) is a mesh fixed in the drinking straw, the mesh diameter of which is smaller than the smallest diameter of the formulation particles (4).

13. A dosage form according to claim 5, characterised in that the barrier device (2) consists of a narrowing of the drinking straw, wherein the cross-section of the drinking straw is narrower than the smallest diameter of the formulation particles (4).

14. A dosage form according to claim 1, characterised in that the drinking straw (1) is rigid or flexible, straight or angled and/or preferably reversibly bendable.

15. A kit for a dosage form for the oral administration of active ingredients, vitamins and/or nutrients comprising a drinking straw (1), which has two orifices (6, 7) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation, and the active ingredient, vitamin and/or nutrient formulation (4) arranged in the drinking straw between the barrier device and the closing device, characterised in that, as the closing device, a cap (3) which closes the drinking straw orifice (6) facing the formulation comprises a bottom (5) made of plastics or metal, preferably aluminium, which is removable by impact and comprises a weakening line at least partially around the entire outer periphery thereof and a physiologically unproblematic conveying liquid which optionally contains active ingredients, nutrients and/or vitamins.

16. A kit according to claim 15, characterised in that the conveying liquid is an aqueous liquid.

17. A kit according to claim 15, characterised in that the conveying liquid is water, lemonade, fruit juice without fruit pulp, tea or coffee.

18. A process for the production of a dosage form for the oral administration of active ingredients, vitamins and/or nutrients comprising a drinking straw (1), which has two orifices (6, 7) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation, and the active ingredient, vitamin and/or nutrient formulation (4) arranged in the drinking straw between the barrier device and the closing device, characterised in that, as the closing device, a cap (3) which closes the drinking straw orifice (6) facing the formulation comprises a bottom (5) made of plastics or metal, preferably aluminium, which is removable by impact and comprises a weakening line at least partially around the entire outer periphery thereof characterised in that the barrier device (2) is inserted into the drinking straw (1), the formulation (4) is introduced into the drinking straw (1) through one drinking straw orifice (6) and this drinking straw orifice (6) is provided with the closing device (3).

19. A process for the production of a dosage form for the oral administration of active ingredients, vitamins and/or nutrients comprising a drinking straw (1), which has two orifices (6, 7) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation, and the active ingredient, vitamin and/or nutrient formulation (4) arranged in the drinking straw between the barrier device and the closing device, characterised in that, as the closing device, a cap (3) which closes the drinking straw orifice (6) facing the formulation comprises a bottom (5) made of plastics or metal, preferably aluminium, which is removable by impact and comprises a weakening line at least partially around the entire outer periphery thereof characterised in that one drinking straw orifice (6) is provided with the closing device (3) and the formulation (4) and the barrier device (2) are introduced into the drinking straw (1) through the drinking straw orifice (10).

20. A process for the production of a dosage form according to claim 18, characterised in that at least one end of the drinking straw is provided with a removable protective cap with a bottom made from impact-resistant material.
